(19) United States
(12) Patent Application Publication
(10) Pub. No.: US 2012/0302507 A1
(43) Pub. Date: Nov. 29, 2012

(54) Liquid Product of Botulinum Toxin Type A

(76) Inventor: Jong Wook HAM, Seoul (KR)

(21) Appl. No.: 13/452,343

(22) Filed: Apr. 20, 2012

(30) Foreign Application Priority Data
May 25, 2011 (KR) 10-2011-0049424

(51) Int. Cl. A61K 38/16; C07K 14/33; A61P 23/02

(52) U.S. Cl. 514/18.1; 530/350

(57) ABSTRACT

Disclosed are a liquid product of botulinum toxin type A and a method for conserving the potency of botulinum toxin type A using a dextrose solution. Free of a stabilizer, such as albumin or gelatin, the liquid product of botulinum toxin type A completely excludes the possibility of cross infections such as AIDS and bovine spongiform encephalopathy. In addition, botulinum toxin type A is preserved as a liquid product in combination with a dextrose solution and can be clinically used as is, without the aid of physiological saline. Therefore, the liquid product enjoys the advantage of being convenient for use and avoiding a decrease in the potency as occurs upon dilution with physiological saline. Serving as a natural preserving and stabilizing agent, the dextrose solution allows botulinum toxin type A to be stored and distributed in the form of liquid phase over a long period of time and conserves the potency of the toxin at a constant level, which in turn guarantees constant clinical results.

### BACKGROUND OF THE INVENTION

1. Field of the Invention

The present invention relates to a liquid product of botulinum toxin type A and a method for conserving the potency of botulinum toxin type A. More particularly, the present invention relates to the use of a dextrose solution in conserving the potency of botulinum toxin type A.

2. Description of the Related Art

Botulinum toxin is a protein produced by the bacterium Clostridium botulinum. It binds irreversibly to presynaptic nerve endings to inhibit the release of acetylcholine at a neuromuscular junction, thus blocking muscular contraction. This activity is now being diverted to relax muscles for treatment purposes. Meanwhile, within two days after a muscle has been exposed to the toxin, the axon terminal begins to allow the appearance of new external unmyelinated collateral sprouts, which in turn form new neuromuscular junctions at surrounding muscle fibers.

For this reason, the maintenance of a desired clinical effect of the toxin requires an injection every three˜six months. To date, botulinum toxin is used for treating many neuromuscular diseases thanks to its pharmaceutical properties, and the application thereof to various fields has been expanding.

There are eight serologically distinct toxin types (A, B, C1, C2, D, E, F and G) of which type A is the most potent. The present invention addresses a novel liquid product of botulinum toxin type A and a method for conserving the potency thereof.

Botulinum toxin is the most powerful natural biological agent yet discovered. A controlled administration of botulinum toxin is utilized to provide muscle paralysis for the treatment of neuromuscular disorders characterized by excessively hypersensitive skeletal muscles. The main conditions treated with botulinum toxin type A are facial spasm, adult-onset spasmodic torticollis, anal fissure, blepharospasm, cerebral palsy, cervical dystonia, migraine, strabismus, temporomandibular disorder and various muscle spasm symptoms. Other uses of botulinum toxin type A include prevention of wrinkles, treatment of facial spasm or contractions and excessive sweating, and plastic surgery.

As a therapeutic agent for such clinical conditions, a product of botulinum toxin, particularly, botulinum toxin type A, typically employs a stabilizer, such as albumin or gelatin, and is marketed in a lyophilized powder form to prolong its shelf life.

For clinical application, however, the conventional powdered product must be diluted with physiological saline and loaded into a syringe.

Thus, conventional products are inconvenient for clinical use and are likely to be contaminated or to commit an error in the course of diluting with physiological saline. In addition, the employment of albumin which is the protein extracted from human serum or gelatin which is the protein extracted from animals such as cattle, may induce the likelihood (although to a small degree) of cross infection by a virus or a transmissible agent responsible respectively for AIDS and bovine spongiform encephalopathy. Further, a higher protein content may allow an increased possibility of antibody formation or allergic response, so that conventional products cannot guarantee 100% of clinical safety to the patients.

Moreover, because the dilution of conventional products with physiological saline causes a decrease in the potency of the toxin (decreased by 50% upon dilution), the liquefied toxin should be used within four hours after dilution. Also, such an unstable potency leads to non-constant clinical results.

To overcome the above-mentioned problems, Korean Patent Application No. 10-2008-0049914 discloses “Stable Liquid Composition of Botulinum Toxin”. This composition is expensive because it contains various ingredients such as polysorbate 20 and methionine, and optionally isoleucine in addition to botulinum toxin. Further, polysorbate 20 is an environmental hormone, so the composition is thought to be difficult to produce as a product.

### SUMMARY OF THE INVENTION

Accordingly, the present invention has been made keeping in mind the above problems occurring in the prior art, and an object of the present invention is to provide a method for conserving a potency of botulinum toxin type A at a level of 100% without decreasing its potency for a long period of time by using a dextrose solution which is harmless to human body as a stabilizer and preservative.

Another object of the present invention is to provide a liquid product of botulinum toxin type A which contains no protein such as albumin or gelatin as a stabilizer and excludes the possibility of cross contamination by viruses or transmissible agents.

A further object of the present invention is to provide a liquid product of botulinum toxin type A comprising a dextrose solution which is harmless to human body as a stabilizing and preserving agent instead of polysorbate 20, a harmful environmental hormone.

Still a further object of the present invention is to provide a liquid product of botulinum toxin type A comprising a dextrose solution as a preservative and stabilizing agent which can be stored and distributed for a long period of time at 2˜8° C. without decreasing its potency in the liquid phase and which is advantageous for commercialization.

Leading to the present invention, intensive and thorough research into the commercialization of botulinum toxin type A, conducted by the present inventor, resulted in the finding that a dextrose solution, innocuous to the human body, is very useful as a stabilizing and preserving agent and can conserve the potency of botulinum toxin type A for a long period of time.

The present invention is characterized by the addition of a dextrose solution to botulinum toxin type A.

For use as a stabilizing and preserving agent the dextrose solution has a concentration of from 6 to 12% and preferably from 8 to 10%.

The liquid product of botulinum toxin type A according to the present invention may be prepared by mixing, dissolving and diluting botulinum toxin type A with dextrose solution in a vessel in such a way that the dextrose solution is added to slowly flow down to be contacted with the inner wall of the vessel containing botulinum toxin type A.

In the liquid product of botulinum toxin type A according to the present invention, the dextrose solution is contained in an amount of from 2 to 6 ml, and preferably in an amount of from 3 to 5 ml per 100 units of botulinum toxin type A.

Optionally, the liquid product of botulinum toxin type A according to the present invention may further comprise an anesthetic for mitigating pain during treatment with the botulinum toxin type A, the anesthetic being selected from among lidocaine, tetracaine, dibucaine, provacaine and bupivacaine.

According to the present invention, the liquid product of botulinum toxin type A may be prepared into a one-component system in which dextrose solution is added to botulinum toxin type A in the manufacturing step of botulinum toxin type A, pre-mixed and packed together; may be prepared into a two-component system in which botulinum toxin type A and dextrose solution are each packed separately and adapted to be mixed prior to or during application; or may be prepared by diluting a conventional botulinum toxin type A product (e.g., Botox) with the dextrose solution, whereby the potency of botulinum toxin type A can be conserved for a long period of time. Also, according to the present invention, the liquid product of the present invention is characterized in that the liquid product remaining after use can be also re-used without decreasing its potency for a long period of time.

As described hitherto, the liquid product of botulinum toxin type A according to the present invention can retain the potency of botulinum toxin type A at a level of 100% under the protection of the dextrose solution for a long period of time (12˜15 months) without any degradation.

Absence of a protein such as albumin or gelatin as a stabilizer, completely excludes the possibility of cross infections such as AIDS and bovine spongiform encephalopathy.

In the present invention, botulinum toxin type A is preserved as a liquid product in combination with a dextrose solution and can be clinically used as it is, without the aid of physiological saline. Therefore, the liquid product of the present invention enjoys the advantage of being convenient to use and avoiding a decrease in the potency as occurs upon dilution with physiological saline in the prior art.

Serving as a natural preserving and stabilizing agent, the dextrose solution useful in the present invention allows botulinum toxin type A to be stored and distributed in the liquid phase for a long period of time (12˜15 months) at 2˜8° C. and conserves the potency of the toxin at a constant level, which in turn guarantees constant clinical results.

In addition, the liquid product of botulinum toxin type A according to the present invention is economically beneficial because it has a simple composition and needs not the use of a solvent such as physiological saline.

According to the present invention, the liquid product of botulinum toxin type A may be prepared into a one-component system in which dextrose solution is added to botulinum toxin type A in the manufacturing step of botulinum toxin type A, pre-mixed and packed together; may be prepared into a two-component system in which botulinum toxin type A and the dextrose solution are each packed separately and adapted to be mixed prior to or during application; or may be prepared by diluting a conventional botulinum toxin type A product (e.g., Botox) with the dextrose solution, whereby the potency of botulinum toxin type A can be conserved for a long period of time. Also, according to the present invention, the liquid product of the present invention is characterized in that the liquid product remaining after use can be also re-used without decreasing its potency for a long period of time.

### DESCRIPTION OF THE PREFERRED EMBODIMENTS

The present invention addresses a conservation of the potency of botulinum toxin type A. More particularly, the present invention pertains to a liquid product of botulinum toxin type A which retains the potency of the toxin for a long period of time without degradation by using a dextrose solution, and the use of a dextrose solution in conserving the potency of botulinum toxin type A.

The liquid product of botulinum toxin type A of the present invention comprises botulinum toxin type A and a dextrose solution. Optionally, it may further comprise an anesthetic such as lidocaine, tetracaine, dibucaine, provacaine or bupivacaine, in order to mitigate the pain upon the injection of the liquid product of the present invention.

Dextrose included in the liquid product of the present invention, is harmless to the human body and is used in infusion solutions. Because the dextrose solution useful in the present invention is made isotonic to human fluid, it may reduce pain when injected and may prevent injury of muscle tissue. Functioning to stabilize and preserve proteins as a natural material, dextrose allows the liquid product of botulinum toxin type A to be stored and distributed for a long period of time without degrading the potency of the botulinum toxin type A.

Also, the liquid product of botulinum toxin type A of the present invention requires no stabilizers such as albumin or gelatin, which are used to prolong the shelf life of conventional products of botulinum toxin type A, thus excluding the possibilities of cross infections from such additives. Further, the liquid product of botulinum toxin type A of the present invention does not need to be diluted with physiological saline, so it can avoid completely the decrease of the potency and the contamination occurring in the course of the dilution.

The dextrose solution useful in the present invention ranges in concentration from 6 to 12% and preferably from 8 to 10%. Its content in the liquid product of botulinum toxin type A of the present invention is on the order of from 2˜6 ml and preferably on the order of from 3 to 5 ml per 100 units of botulinum toxin type A.

Generally, a commercial product of botulinum toxin is in freeze-dried form. The manufacturers recommend that botulinum toxin type A be clinically applied within 4 hours after being diluted with 0.9% physiological saline, the reason being that the potency of botulinum toxin type A decreases with time. In practice, however, because botulinum toxin type A is expensive and is used in a very small amount per single patient, the remainder is stored in a refrigerator and is re-used within 1˜2 weeks. In full consideration of this practical situation, the present invention provides a liquid product comprising botulinum toxin type A combined only with a dextrose solution, which requires no dilution steps using physiological saline in the prior art and conserves the potency of botulinum toxin type A at a level of 100% without any degradation for one year or longer (e.g., 12˜15 months).

A better understanding of the present invention may be obtained through the following examples which are set forth to illustrate, but are not to be construed as limiting the present invention.

### Example 1

### Preparation of Liquid Product of Botulinum Toxin Type A

Botulinum toxin type A was homogeneously mixed and dissolved with a dextrose solution. For use in mouse tests, the dextrose solution having a concentration of 10% was added in an amount of 5 ml per 100 units of botulinum toxin type A to prepare a liquid product of botulinum toxin type A.

(Assay for Potency of Liquid Product of Botulinum Toxin Type A)

At predetermined times after being prepared as described above, the liquid product of botulinum toxin type A according to the present invention was assayed for potency. The potency of botulinum toxin is expressed in unit. One unit of botulinum toxin corresponds to a lethal dose (LD50) for one mouse. That is, intraperitoneal injection of two units of botulinum toxin kills a mouse weighing 20 g.

Immediately, 2, 4, 8, 16, 24, 48 and 60 weeks after preparation, the liquid product of botulinum toxin type A was intraperitoneally injected at a dose of 2 units (0.2 ml) into each of 10 mice. In all cases, the liquid product was found to preserve the potency at a level of 100% without degradation. The remainder of the liquid product also experienced no potency degradation as revealed by assaying in the same manner.

This surprising result is attributed solely to the dextrose solution that acts as a stabilizing and preserving agent in the liquid product of botulinum toxin type A.

### Example 2

The effect of the liquid product of botulinum toxin type A according to the present invention was compared to a conventional one. For this, mice were divided into four groups of 10: control 1, test group 1 (conventional), test group 2 (inventive), and control 2. The effect was assayed by means of the death toll 72 hours after injection per each group.

(Test 1. Intraperitoneal Injection at Dose of 1 Unit)

Each of the mice was intraperitoneally injected with 0.1 ml of physiological saline for control 1, with 1 unit (0.1 ml) of Botox diluted with physiological saline for test group 1, with 1 unit (0.1 ml) of the botulinum toxin type A diluted with 10% dextrose solution for test group 2 and with 0.1 ml, of 10% dextrose solution for control 2. At 72 hours after injection, the death toll was zero (0) in control 1, test group 1 and control 2 and four in test group 2.

(Test 2. Intraperitoneal Injection at Dose of 2 Units)

Each of the mice was intraperitoneally injected with 0.2 ml of physiological saline for control 1, with 2 units (0.2 ml) of Botox diluted with physiological saline for test group 1, with 2 units (0.2 ml) of the botulinum toxin type A diluted with 10% dextrose solution for test group 2 and with 0.2 ml of 10% dextrose solution for control 2. At 72 hours after injection, the death toll was zero (0) in control 1 and control 2, four in test group 1, and ten in test group 2.

(Test 3. Intraperitoneal Injection at Dose of 2 Units of the Solutions used in Test 0.2 after Storage at 4° C. for 2 Weeks)

Each of the mice was intraperitoneally injected with 0.2 ml of physiological saline for control 1, with 2 units (0.2 ml) of Botox diluted with physiological saline for test group 1, with 2 units (0.2 ml) of the botulinum toxin type A diluted with 10% dextrose solution for test group 2 and with 0.2 ml of 10% dextrose solution for control 2. At 72 hours after injection, the death toll was zero (0) in control 1, test group 1 and control 2, and ten in test group 2.

### Example 3

### (Comparison Test on Treatment Effect of Forehead Wrinkles)

Application region: A commercial product (Solution obtained by diluting conventional Botox with 2 ml of physiological saline) was injected into the forehead at two injection points. One point was 2.5 cm above away from the inner upper edge of the right eyebrow while the other was established 3 cm outside away from the above point. The liquid product of the present invention (Solution obtained by diluting botulinum toxin type A with 4 ml of 10% dextrose solution) was injected into the corresponding points for the left eyebrow.

Injection: The commercial product (Solution obtained by conventional Botox with 2 ml of physiological saline) was injected at a dose of 2.5 units (0.05 ml) for each of the right injection points in such a manner that the syringe needle was inserted vertically to a depth of 2˜3 mm below the skin surface. On the other hand, the liquid product of the present invention (Solution obtained by diluting botulinum toxin type A with 4 ml of 10% dextrose solution) was injected at a dose of 1.25 units (0.05 ml) for each of the left injection points to the same depth (primary injection). Two weeks after the injection, treatment effects on the left and the right forehead wrinkles were compared, and the results of the primary injection are summarized in Table 1.

Six months later, the commercial product (Solution obtained by diluting conventional Botox with 2 ml of physiological saline) was injected at a dose of 2.5 units (0.05 ml) into each of the right injection points. On the other hand, after the primary injection of the liquid product of the present invention, the remainder was stored at 4° C. for six months and injected at a dose of 1.25 units (0.05 ml) into each of the left injection points (secondary injection). Two weeks after the secondary injection, treatment effects were compared between the left and the right forehead wrinkles. The results of the secondary injection (6 months after the primary injection) are summarized in Table 1.

Six months after the secondary injection (that is, 12 months after the primary injection), the commercial product (Solution obtained by diluting conventional Botox with 2 ml of physiological saline) was injected at a dose of 2.5 units (0.05 ml) into each of the right injection points. On the other hand, after the primary injection of the liquid product of the present invention, the remainder was stored at 4° C. for 12 months and injected at a dose of 1.25 units (0.05 ml) into each of the left injection points (tertiary injection). Two weeks after the tertiary injection, treatment effects were compared between the left and the right forehead wrinkles. The results of the tertiary injection (12 months after the primary injection) are summarized in Table 1.

TABLE 1

| | | | | Comparison |
|---|---|---|---|---|
| | | | | on Treatment |
| | | | | Effects |
| | | | Treatment | between |
| | Solution for | Solution for | Effect on | Left and |
| | Right | Left | Right | Right |
| | Forehead | Forehead | Forehead | Forehead |
| Primary | Commercial | Inventive | Good | The same |
| Injection | product | Product | vanishing | |
| | (solution | (solution | of wrinkles | |
| | obtained by | obtained by | | |
| | diluting | diluting | | |
| | conventional | botulinum | | |
| | Botox with | toxin type A | | |
| | 2 ml of | with 4 ml of | | |
| | physiological | 10% dextrose | | |
| | saline) | solution) | | |
| Secondary | The same as | Remainder | Good | The same |
| Injection | above | after | vanishing | |
| (6 months | | primary | of wrinkles | |
| after | | injection | | |
| primary | | | | |
| injection) | | | | |
| Tertiary | The same as | Remainder | Good | The same |
| Injection | above | after | vanishing | |
| (12 months | | primary | of wrinkles | |
| after | | injection | | |
| primary | | | | |
| injection) | | | | |

Wrinkle vanishing effects were divided into 4 classes: no effects, insufficient effect, good effect and excessive effect.

### DISCUSSION

1. The same treatment effects were observed between the left and the right forehead although the left was treated with 50% of the potency used for the right. On the whole, 1.25 units of conventional Botox are insufficient to bring about the desired treatment effect, but the liquid product diluted with 10% dextrose solution of the present invention caused forehead wrinkles to vanish significantly, indicating that the dextrose solution is a potent stabilizer capable of conserving 100% of the potency of botulinum toxin type A as it is.

2. Even after being stored for 6 and 12 months, the liquid product diluted with 10% dextrose solution of the present invention showed constant effects, indicating that the dextrose solution functions as a preservative as well as a stabilizer. This is the essential point of the present invention.

3. Conventional products decrease their potency by 50% as soon as they are diluted with physiological saline. The potency decreases with time and becomes clinically useless after two weeks have passed.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A liquid product of botulinum toxin type A, retaining a potency of botulinum toxin type A at a level of 100% without decreasing its potency for 12˜15 months, prepared by mixing, dissolving and diluting botulinum toxin type A with dextrose solution in a vessel in such a way that the dextrose solution is added to slowly flow down to be contacted with the inner wall of the vessel containing botulinum toxin type A in an amount of from 2 to 6 ml per 100 units of botulinum toxin type A, said dextrose solution having a concentration of 6˜12%.

2. The liquid product of botulinum toxin type A of claim 1, further comprising an anesthetic selected from among lidocaine, tetracaine, bupivacaine, provacaine and dibucaine.

3. The liquid product of botulinum toxin type A of claim 1, being in a form of a two-component system in which botulinum toxin type A and the dextrose solution are each packed separately and adapted to be mixed prior to or during application.

4. Use of a dextrose solution as a stabilizing and preserving agent for botulinum toxin type A.
